(19) United States
(12) Patent Application Publication
(10) Pub. No.: US 2003/0106551 A1
(43) Pub. Date: Jun. 12, 2003

(54) Resistive heater formed inside a fluid passage of a fluid vaporizing device

(76) Inventors: F. Murphy Sprinkel JR., Glen Allen, VA (US); Jay A. Fournier, Richmond, VA (US)

Correspondence Address: Peter K. Skiff, BURNS, DOANE, SWECKER & MATHIS, L.L.P., P.O. Box 1404, Alexandria, VA, 22313-1404, US

(21) Appl. No.: 10/003,437

(22) Filed: Dec. 6, 2001

(51) Int. Cl. A61M 15/00; A61M 16/00

(52) U.S. Cl. 128/203.16; 128/203.26

(57) ABSTRACT

A fluid vaporizing device such as an aerosol generator having a tubular heater in the form of thin film of resistance heating material lining a fluid passage and method for forming the tubular heater within the fluid passage. The tubular heater can be heated to volatilize a fluid within the passage. The fluid vaporizing device can be used for the generation of aerosols containing medicated material.

### BACKGROUND OF THE INVENTION

1. Field of the Invention

The present invention relates to an apparatus and method for forming a resistive heater within a fluid passage of a fluid vaporizing device such as an aerosol generator. The present invention is particularly useful for the generation of aerosols containing medicated material.

2. Description of the Related Art

Aerosols are gaseous suspensions of fine solid or liquid particles and are useful in a wide variety of applications. For example, medicated liquids and powders may be administered in aerosol form. Such medicated aerosols include, for example, materials which are useful in the treatment of respiratory ailments, in which case the aerosols may be inhaled into a patient's lungs. Aerosols may also be used in non-medicinal applications including, for example, dispensing air fresheners and insecticides and delivering paints and/or lubricants.

In aerosol inhalation applications, it is typically desirable to provide an aerosol having an average mass median particle diameter of less than 2 microns to facilitate deep lung penetration. In certain applications, it is generally desirable to deliver medicated material at high flow rates, for example, above 1 mg per second. Propellant driven aerosol generators suited for delivering medicated material are incapable of delivering material at such high flow rates while maintaining a suitable average mass median particle diameter. In addition, such aerosol generators deliver an imprecise amount of aerosol compared with the amount of aerosol that is intended to be delivered.

Commonly assigned U.S. Pat. No. 5,743,251, the entire contents of which document are hereby incorporated by reference, discloses an aerosol generator which forms an aerosol by volatilizing a liquid in a capillary tube and delivering the vapor to a mouthpiece for inhalation by a user of the device. The fluid can be heated by passing electrical current through a heater wire coiled around a ceramic capillary tube or through a thin platinum layer deposited on the outside of a ceramic capillary tube. The design of this heating arrangement requires heat to travel from the heater through the ceramic capillary tube in order to heat the fluid in the capillary tube to a temperature sufficient to volatilize the fluid.

In light of the foregoing, there exists a need in the art for an economical method for manufacturing a fluid vaporizing device such as an aerosol generator and a fluid vaporizing device made by such a method. There is also a need for an improved heater design which permits more efficient heating of the fluid by locating the heater inside the fluid passage of a fluid vaporizing device such as an aerosol generator useful in an inhaler device.

### SUMMARY OF THE INVENTION

The invention provides a fluid vaporizing device which includes a tubular heater comprising a thin electrically resistive film lining at least part of an interior surface of a fluid passage. The thin electrically resistive film may act as a heater when electrical energy is passed through the film so as to volatilize a fluid in the passage.

The invention also provides a method of making the fluid vaporizing device by forming the tubular heater within the fluid passage and providing electrically conductive contacts which allow an electrical current to pass through the tubular heater and heat the fluid in the fluid passage.

The fluid vaporizing device can be used to generate a vaporized fluid such as an aerosol of a medicated liquid by supplying fluid to the fluid passage, heating the tubular heater so as to volatilize the fluid in the fluid passage, and directing the volatilized fluid out of the fluid passage via an outlet.

### BRIEF DESCRIPTION OF THE DRAWINGS

The objects and advantages of the invention will become apparent from the following detailed description of the preferred embodiments thereof in connection with the accompanying drawings, in which:

FIG. 1 is an exploded view of an exemplary fluid passage assembly for a fluid delivery device such as an aerosol generator in accordance with the invention.

FIG. 2 is an exploded view of an exemplary aerosol generator in accordance with the invention; and

FIGS. 3-4 show exploded views of alternative aerosol generators in accordance with the invention.

FIG. 5 shows an alternative fluid passage assembly in accordance with the invention.

FIG. 6 shows an alternative aerosol generator in accordance with the invention.

### DETAILED DESCRIPTION OF THE PREFERRED EMBODIMENTS OF THE INVENTION

The invention provides a fluid vaporizing device such as an aerosol generator which includes a tubular thin film heater lining all or a portion of the length of a fluid passage, the heater being operable to volatilize fluid in the passage. With such an arrangement, the heater can form at least part of a surface defining the flow passage. As such, heat generated by the heater can be directly transferred to the fluid to maximize thermal efficiency of the heater. The heater can be formed within the fluid passage after the fluid passage is formed in a monolithic or multilayer body made of metal, polymer and/or ceramic materials. By depositing the thin film heater within a fully enclosed fluid passage, the manufacturing steps and/or costs can be reduced and the heater can be provided over a maximum surface area of the fluid passage which preferably is of capillary size and/or over complex fluid passage geometries.

The fluid passage is preferably formed in a fluid passage body such as a single or multilayer ceramic or glass body. The passage has an enclosed volume opening to an inlet and an outlet either of which may be open to the exterior of the fluid passage body or may be connected to another passage within the same body or another body or to fittings. The thin film heater is “tubular” in that it coats or lines the interior of a fluid passage such that the heater has an inlet and an outlet and encloses a volume through which a fluid may pass. One surface of the thin film is in contact with the interior of the fluid passage body. The other surface of the thin film heater may define all or part of the interior of the fluid passage or may itself be thinly coated by an electrically non-conductive material; such a coating may then define all or part of the interior of the fluid passage. The fluid passage and therefore the heater may be any shape comprising an enclosed volume opening to an inlet and an outlet and through which a fluid may pass. The fluid passage may have any desired geometry. A preferred geometry is a round hole of uniform diameter with an especially preferred hole size of capillary dimensions (e.g., 0.01 to 10 mm, preferably 0.05 to 1 mm, more preferably 0.1 to 1 mm). Alternatively, the capillary passage can be defined by transverse cross sectional area of the passage which can be 8×10⁻⁵ to 80 mm², preferably 2×10⁻³ to 8×10⁻¹ mm² and more preferably 8×10⁻³ to 2×10⁻¹ mm². Other fluid passage geometries include non-circular holes such as triangular, square, rectangular, oval or other shape and the cross section of the hole need not be uniform. The fluid passage can extend rectilinearly or non-rectilinearly and may be a single fluid passage or multi-path fluid passage. The “tubular heater” according to the invention thus encircles at least a portion of the flow path through which fluid passes as it is heated by the heater.

The present invention provides a method for forming a thin film of resistive material on the inside walls of a fluid passage. The thin resistive film may function as a heater and may be in direct contact with a fluid in the passage. Thus, the thin resistive film may be used to volatize a liquid in the passage, thereby ejecting the fluid as a vapor therefrom.

The thin film heater can be formed by any suitable technique such as electroless deposition, electrochemical deposition, washing with resistive/conductive inks, ceramic circuit printing, vapor deposition, and thermal decomposition of a metal salt deposited in solution whereby a thin resistive layer is created within a formed passage. A preferred method comprises contacting the interior of a fluid passage with a metal salt solution, most preferably a 0.1 to 5% solution of chloroplatinic acid (H₂PtCl₆), boiling off the residual fluid, and thermally decomposing the deposited salt to platinum (Pt) metal in a furnace at 115 to 1000° C., preferably 300 to 600° C., most preferably about 500° C. The method can be used to provide a tubular heater in an inhaler having a fluid passage of capillary size.

According to an aspect of the present invention, a fluid vaporizing device in the form of an aerosol generator includes a fluid passage and a tubular heater comprising a thin resistive film lining at least a portion of the fluid passage, the thin resistive film being introduced within the formed passage by a deposition process. Fluid supplied by a fluid supply can be vaporized by the heater and an outlet of the passage can direct fluid out of the fluid passage into ambient air so as to form an aerosol via condensation of the vapor.

The fluid vaporizing device preferably includes electrical contacts for supplying electrical power to the tubular heater within the passage. In a preferred embodiment, the fluid passage is formed in a ceramic body and the contacts comprise copper posts bonded to the ceramic body by diffusion of copper oxide on the surface of the posts into the ceramic material. The tubular heater can be formed by depositing a thin resistive film in the fluid passage such that the film is in direct contact with the ends of the copper posts. However, electrical current can be delivered to the deposited heater by any suitable arrangement, e.g., a screen printed electrically conductive circuit could be formed on a green ceramic body having the fluid passage therein and the assembly could be low temperature cofired ceramic construction.

In one embodiment of the invention, the fluid passage is formed by the interior of a non-conductive capillary tube of glass, ceramic or any other material with appropriate chemical and heat resistance properties such as, for example, a polymer, resin, or composite material. Alternatively, the fluid passage may be formed by molding, casting or machining in any suitable material. Further, the passage may be provided by coating a formed passage in heat resistant material, including a conductive material such as stainless steel, with a non-conductive coating and the heater can comprise a thin resistive layer formed in the interior of the coated passage.

In a preferred embodiment of the present invention, the fluid passage is arranged between a first layer and a second layer, wherein the first and second layers at least partially define the fluid passage, and wherein the fluid passage is formed prior to the formation of a thin resistive heating element therein. In another embodiment, the passage comprises a channel or opening formed in a third layer disposed between the first and second layers. The layers can comprise low temperature cofired ceramic (LTCC) material.

The fluid vaporizing device can be used to generate a vapor or aerosol by supplying fluid to the passage while heating the heater sufficiently to vaporize the fluid. For example, the vapor can be directed from an outlet of the passage into ambient air and an aerosol with a desired droplet size can be obtained via condensation of the vapor. However, the vapor can be used for other purposes such as effecting chemical reactions, depositing coatings, etc.

The fluid vaporizing device can be made in an economical manner by depositing the tubular heater in an already formed fluid passage. For example, a fluid passage can be formed in an electrically insulating material and the tubular heater can be formed by depositing a resistive film in the previously formed passage. To provide power to the heater, electrical connections can be added which allow electrical current to flow through the heater and heat the heater to a temperature sufficient to volatilize a fluid supplied to the passage.

The invention will now be explained with reference to the drawings wherein like reference numerals designate identical or corresponding elements throughout the several figures.

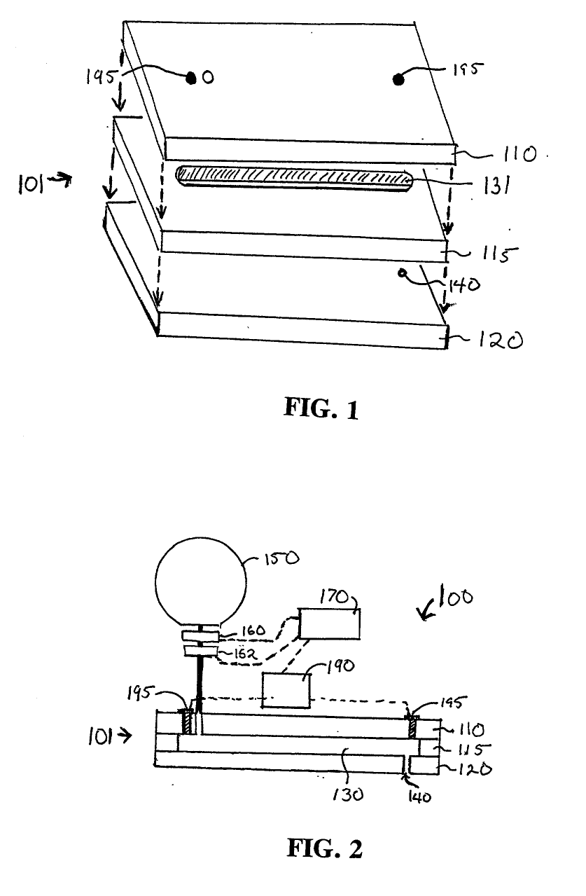

FIGS. 1 and 2 illustrate a fluid vaporizing device which can be used as an aerosol generator 100 according to one aspect of the present invention. The device 100 heats fluid within a fluid passage assembly 101 and directs volatilized fluid out of an outlet passage 140 of the fluid passage assembly and away from the device 100. The device 100 can be used to eject a volatilized fluid such as a solution containing a medicated material into an ambient air atmosphere such that the volatilized fluid condenses in the atmosphere and forms an aerosol with a desired median droplet/particle size, e.g., 0.1 to 2 μm.

The present invention provides a method for forming a tubular heater within a fluid passage 130 in a fluid passage assembly 101 thus simplifying the manufacture of the fluid vaporizing device. The tubular heater can be deposited as a thin film on the entire interior of the fluid passage and may be in direct contact with fluid in the fluid passage thereby increasing the efficiency of the volatilization.

The fluid may include any material capable of volatilization by the aerosol generator 100. In a preferred embodiment, the fluid does not decompose when exposed to the heat required for volatilization thereof. For inhaler applications, a preferred fluid is a medicated material such as, for example, a material that is useful in the treatment of respiratory ailments. In such applications, the volatilized fluid is ejected from an inhaler as an aerosol which can be inhaled into a user's lungs. Alternatively, the fluid may include a non-medicated material used in non-inhaler applications in which an aerosol may or may not be formed such as aroma generation, coating applications, spray applications, etc.

Referring to FIG. 1, a preferred embodiment of a fluid passage assembly 101 for an aerosol generator 100 includes a first layer 110, a second layer 120 and third layer 115 between the first and second layers with a void 131 cut into it. When bonded together as shown in FIG. 2, layers 110, 115, and 120 define the fluid passage 130. The three layers 110, 115, 120 are preferably formed from a heat-resistant material that is capable of withstanding the temperatures and pressures generated in the fluid passage 130 and the assembly is preferably capable of withstanding repeated heating cycles. Also, the heat-resistant material preferably does not react with the fluid contained in the fluid passage 130. The heat-resistant material may include, for example, ceramic materials such as alumina, zirconia, silica, aluminum silicate, titania, yttria-stabilized zirconia, magnesia or mixtures thereof, preferably alumina. According to a preferred embodiment of an aerosol generator of an inhaler, each layer can have a length of from about 1 to 100 mm, e.g., about 15 mm; a width of from about 1 to 100 mm, e.g., about 15 mm; and a thickness of from about 0.001 to 10 mm, e.g., about 0.08 mm.

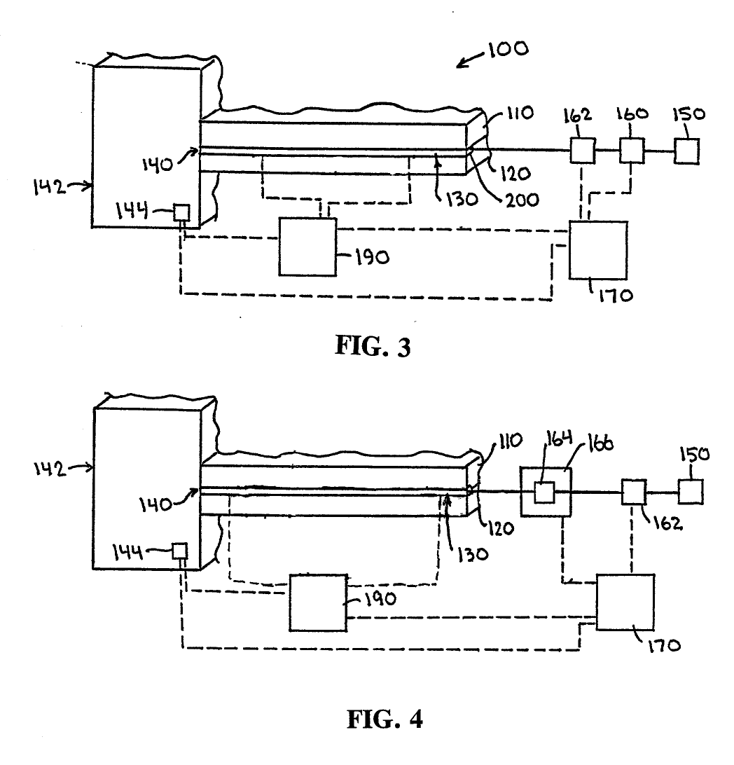

The fluid passage 130 can be formed by any suitable technique, e.g., machining, molding, extrusion, or otherwise forming the passage in a monolithic or multilayer body. For example, channel 200 can be formed into a first and/or second layers, as shown in FIGS. 3 and 4. In FIG. 3, the channel 200 is formed in layer 120 and the first and second layers 110, 120 are attached together, thereby enclosing the passage 130 therebetween. In this manner, the channel 200 of the second layer 120 and the first layer 110 define the fluid passage 130. A further channel may optionally be disposed upon the side of the first layer 110 that is attached to the second layer 120, wherein such additional channel further defines the fluid passage 130, as shown in FIG. 4. The additional channel is preferably arranged such that the additional channel and the channel 200 form a single fluid passage 130 when the first and second layers 110, 120 are attached together.

The aforementioned first, second and third layers 110, 120, 115 may be attached together using various techniques, including, for example, adhesive bonding. The adhesive material used to attach the layers is preferably capable of withstanding repeated heating cycles and may include, for example, a metal, a cement, an epoxy, an acrylic, a cyanoacrylic or mixtures thereof, preferably an acrylic cement. Alternatively, other techniques may be used to attach the layers 110, 120, 115 together such as, for example, mechanical or metallurgical bonding, e.g., use of a brazing material, glass or filled glass to hold the layers together. A preferred technique is compression and firing of green ceramic layers to form a bonded multilayer structure.

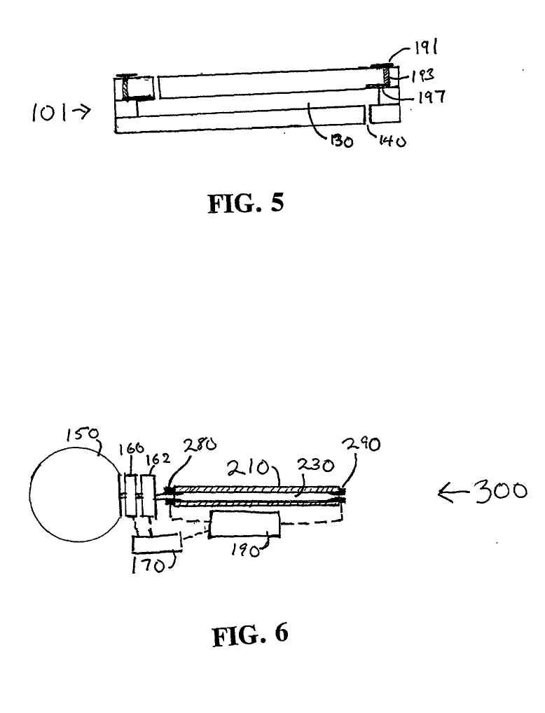

In a preferred embodiment, illustrated in FIGS. 1 and 5, the fluid passage assembly is constructed from three layers of green ceramic about 0.01 mm thick such as GreenTape™ (Part No. 951AT, 3.8 mils thickness) available from E. I. du Pont de Nemours and Company as part of a low temperature cofired ceramic (LTCC) system. Layer 110 includes, for example, an inlet hole and two vias for conductive contacts 195. A second layer 120 includes an outlet hole. A third layer 115, includes a void which defines the sides and end of the passage 130. For aerosol generators of an inhaler, the inlet hole can have any desired size, e.g., a circular hole with a diameter of about 0.01 to 10 mm, preferably 0.1 to 1 mm, the outlet hole can have any desired size, e.g., a circular hole with a diameter of 0.01 to 10 mm, preferably 0.05 to 1 mm, more preferably 0.1 to 0.5 mm and the channel in the middle layer 115 can have any suitable dimensions, e.g., 0.2 mm×12 mm. The Green Tape™ is preferably laminated and fired according to the manufacturer's standard procedures.

The fluid passage 130 may have any desired configuration. For example, the passage can be linear and of uniform cross section to direct flow of the fluid in a particular direction. However, the fluid passage 130 can have a non-linear and/or non-uniform cross section, configuration such as in the case where the direction of fluid flow through the passage 130 contains at least one turn.

Referring to FIG. 2, the upstream end of the fluid passage 130 is connected to receive a fluid in liquid phase from a fluid supply 150. Volatilized fluid exits the downstream end of the fluid passage 130 through outlet 140. The outlet 140 can be oriented to direct the volatilized fluid in a desired direction and/or the outlet 140 can be sized to achieve a desired aerosol particle size distribution. In a preferred embodiment of an aerosol generator for an inhaler, the outlet 140 is the same size or different in size than the width of channel 200 forming the flow passage 130. For example, the outlet 140 can have any desired size, e.g. a circular opening in a surface of the layer 120 with a diameter of about from 0.01 to 10 mm, preferably about 0.1 to 1 mm.

According to an exemplary embodiment of the present invention, the outlet 140 is an orifice disposed on the first or second layer 110, 120 through which the volatilized fluid flows. The outlet 140 may be disposed at an angle, for example, 10 to 160°, with respect to the axis of fluid flow within the fluid passage 130, to direct the flow of the volatilized fluid out of the fluid passage 130 in a desired direction. According to an alternative embodiment, the fluid passage 130 extends through a side wall of the layers 110, 120, and the outlet 140 is defined by the furthest downstream portion of the fluid passage 130. A conduit (not shown) may be connected to receive the volatilized fluid from the outlet 140 to further direct the flow of volatilized fluid in a desired direction. Such a conduit preferably has a diameter of from about 0.2 mm or larger.

In a preferred embodiment, a valve 160 and/or a pump 162 can be used to control the flow of fluid from the liquid supply 150 to the fluid passage 130. The valve 160 and/or the pump 162 may be manually operated. Alternatively, a controller 170 may manipulate the valve 160 and/or the pump 162 based on various parameters including, for example, the amount of time the valve 160 remains in the open position, or the volumetric amount of fluid that is supplied to the fluid passage 130. In this manner, the valve 160 and/or the pump 162 may enable the liquid supply 150 to deliver a predetermined volume of fluid in liquid phase to the fluid passage 130. In an alternative embodiment, the fluid in liquid phase can be contained in a chamber, and a desired amount of the fluid can be delivered to the flow passage 130 by compressing the fluid in the chamber using a piston, e.g., the fluid can be supplied by a syringe pump.

Another mechanism for delivering the fluid is shown in FIG. 4 wherein fluid is supplied, via pump 162 or other suitable arrangement, to a prechamber 164 of a device such as a metering valve 166. Exemplary embodiments of such metering valves are described in commonly owned U.S. patent application Ser. No. 09/479,597 filed on Jan. 7, 2000, the disclosure of which is hereby incorporated by reference. With such an arrangement, the chamber 164 can be filled with a predetermined volume of fluid, preferably an amount sufficient to deliver a single dose of the fluid to the fluid passage 130. Alternatively, a prechamber which includes a heater can be used to drive fluid to the passage 130 by forming a vapor bubble in the prechamber as described in commonly owned U.S. patent application Ser. No. 09/742,395, filed Dec. 22, 2000, the disclosure of which is hereby incorporated by reference.

The liquid supply 150 provides the fluid to be volatilized in liquid phase to the fluid passage 130. The fluid in liquid phase may be stored in the liquid supply 150 at a pressure above atmospheric to facilitate delivery of the fluid to the fluid passage 130. In an exemplary embodiment, the liquid supply 150 comprises a refillable storage chamber formed of a material suitable for containing the fluid to be volatilized. Alternatively, the liquid supply 150 comprises a disposable storage chamber which, upon exhaustion of the fluid, is discarded and replaced by a new storage chamber.

The fluid passage 130 may volatilize fluid continuously or intermittently. For inhaler applications, the fluid passage 130 may have a liquid volumetric capacity of from about 1×10⁻⁶ ml to 0.005 ml. Alternatively, the fluid passage 130 may have a liquid volumetric capacity of greater than about 0.005 ml, preferably from about 0.1 ml to 1.0 ml. In aerosol inhalation applications, the fluid passage 130 may have a liquid volumetric capacity which is sufficient for containing a predetermined amount of fluid that comprises a metered quantity of fluid. However, the passage 130 can be smaller or larger than a desired volume of fluid to be volatilized.

Referring to FIGS. 2-4, the device 100 includes a tubular resistance heater in the form of a deposited film lining fluid passage 130. The heater is arranged to volatilize the fluid present in the fluid passage 130. A power supply 190 provides the energy to heat the thin resistive layer. The power supply 190 may include, for example, a battery. The heater is preferably formed as a thin resistive film lining the interior of the fluid passage 130 and thus in direct contact with the fluid contained in the fluid passage 130. In an alternative embodiment of the present invention, the thin resistive film may be coated with a passive layer, such as polymer or glass which provides a barrier layer between the heater and the fluid to be vaporized.

The heater preferably comprises a film formed from an electrically resistive heating material which is different from the heat-resistant material used to form the layers 110, 115, 120 of the aerosol generator 100. For example, the resistive material may comprise a resistive heating material such as a pure metal, metal alloy or metal compound. For inhaler applications, the heater is preferably formed of a bio-compatible metal such as platinum, gold, nickel, palladium, silver, tin, alloys, or mixtures thereof. For inhaler or other applications, the heater can be formed from a thin film of platinum which has good chemical and heat resistance and a useful resistivity as a thin film. A heater formed of a thin layer of platinum can also have the desirable property of self healing small fractures of the thin layer resulting from repeated heating and cooling cycles.

Using a material for forming the heater which is different from the material used to form the layers 110, 115, 120 allows the resistance through the heater to be adjusted by varying various parameters including, for example, the thickness and material composition of the heater. In this manner, the resistance of the heater and the amount of heat produced by the heater may be adjusted for various applications. Techniques for controlling heater temperature by monitoring the resistance of the heater are disclosed in commonly owned U.S. application Ser. No. 09/742,322, filed Dec. 22, 2000, the disclosure of which is hereby incorporated by reference.

A thin resistive layer which may function as a heater according to the present invention may be deposited within a passage by a number of techniques. According to a most preferred embodiment, a metal salt solution, preferably chloroplatinic acid (H₂PtCl₆) at a concentration of 0.1-5% is introduced into the fluid passage 130 to thoroughly coat the interior of the passage. Residual fluid is boiled away, and the deposited metal salt is thermally decomposed in a furnace at temperatures of 115° C. to 1500° C., or higher, most preferably about 500° C. for about 5 to 600 minutes, preferably 30 to 180 minutes. The furnace can be a vacuum furnace or a non-vacuum furnace having an air atmosphere or controlled atmosphere such as an inert gas atmosphere. The length of time required depends on oven capacity, mass of the fluid passage assembly material, and passage size and an optimal time and temperature can be determined by routine experimentation. Alternatives to chloroplatinic acid include solutions of other platinum salts such as for example [Pt(NH₃)₄]Cl₂, [Pt(NH₃)₄](NO₃)₂ or (NH₄)₂PtCl₆ at a concentration of 0.1-5%.

Other techniques may be employed to deposit a thin layer of resistive material in the fluid passage. For example, a very fine powder of metal or metal oxide such as platinum powder may be suspended in a carrier, preferably an organic solution or a paste, the suspension is introduced into the passage 130 so as to coat the interior of the passage and the carrier solution is removed by firing such that the deposited material is reduced to a thin metal bonded to the inner surfaces of the passage, e.g., heating at 300 to 1500° C. for 120 to 360 minutes.

Alternatively, the resistive material of the heater may be deposited in the formed passage 130 by electroless deposition (plating). The techniques for electroless deposition are well known and widely practiced. For example, U.S. Pat. No. 6,071,554 discloses methods for forming a platinum electrode within a thimble or test-tube shaped ceramic sensor element. U.S. Pat. No. 5,509,557 discloses electroless deposition of a conductive metal onto a dielectric substrate. U.S. Pat. No. 4,259,409 discloses electroless plating processes for glass or ceramic bodies. And, U.S. Pat. No. 3,995,371 discloses a method for a dental procedure wherein a metal such as platinum is electrolessly deposited directly onto teeth. The technique has been reviewed in Electroless Plating: Fundamentals and Applications (1990) Mallory, G. O., and Hajdu, J. B., Eds., American Electroplaters and Surface Finishers Society: Orlando, Fla. These references and references cited therein are incorporated by reference herein in their entirety.

Briefly, the electroless deposition process may include the following steps: (1) the interior of the passage may be cleaned for example by isopropyl alcohol wash and rinsing with de-ionized water; (2) a smooth material such as a plastic polymer may be prepared to receive metal ions by chemical etching followed by a de-ionized water rinse; (3) the interior of the passage may be sensitized by exposure to acidified stannous chloride; (4) the passage interior surface may be catalyzed by exposure to an acidified palladium or platinum chloride solution; and (5) electroless deposition of a metal by exposure of the interior surface of the fluid passage to a solution containing a metal salt and a reducing agent.

According to an alternative embodiment of the present invention, a formed passage may be filled with an electroless deposition solution containing a platinum salt such as for example [Pt(NH₃)₄]Cl₂, PtCl₄, or (NH₄)₂PtCl₆ at a concentration of preferably 10-20 g/l and a reducing agent such as for example hydrazine or sodium hypophospate. A deposition time of 10 to 1000 minutes, preferably 120 to 360 minutes, more preferably 180 to 240 minutes will provide a 1 to 3 μm thick film of platinum. Heating at 300 to 1500° C. for 30 to 180 minutes can be used to anneal and increase the density of the deposited metal.

In alternative embodiment, multiple layers of various metals may be deposited sequentially and subsequently alloyed by diffusion at an elevated temperature such as 300 to 1500° C. in order to produce a thin film with desired properties of chemical resistance, thermal expansion and electrical resistivity.

Conductive and resistive inks used in LTCC may also be used or modified for use in depositing the thin resistive heater of the present invention. Additional alternative techniques may be used to deposit a thin resistive film which can function as a heater inside the channel 130, e.g., vapor deposition of metals heated by passing a current through a wire threaded through the passage. The film can have a resistance on the order of 0.1 to 10 ohms and preferably about 0.65 ohm.

In a preferred embodiment, the heater within the channel 130 is in electrical contact with at least two contacts 195 which pass an electrical current through the heater. In this embodiment, the power supply 190 which provides the electrical current to the heater is in electrical contact with the first and second contacts 195.

The first and second contacts 195 of the heater are preferably formed from a material which has a lower resistance than that of the resistive material of the heater. For example, the first and second contacts 195 typically include copper or a copper alloy such as, for example, phosphor bronze and silicon bronze, and preferably copper or a copper alloy comprising at least 80% copper or a laminate of gold and silver on copper. Use of such materials prevents or reduces the heating of the contacts 195 prior to the heating of the heater. The contacts 195 are sized to be capable of passing an electrical current through the heater. The contacts 195 may be attached to the layers 110, 115, and/or 120 by diffusion bonding to the ceramic material. In this embodiment, a post of clean copper is placed in contact with the ceramic, preferably with a head of the post in contact with the ceramic. The assembly is placed in a quartz chamber within a furnace with the chamber being flushed with nitrogen or other non-reactive gas of high (greater than 99.95% and ideally at least 99.9999%) purity. The temperature is increased at about 30° C./minute to near 1000° C., slowing to 5° C./minute to 1063° C., it remains at about 1063° C. for about 5 minutes and is then cooled again at about 30° C./minute to room temperature. Copper oxide on the surface diffuses into the ceramic and is reduced to copper metal thereby securing the post to the ceramic. This may be performed before forming the thin resistive film within the fluid passage. Alternatively, the contacts may be formed after formation of the thin resistive film within the fluid passage. Alternatively, steps in forming the contacts, such as heating and cooling steps, may be combined with steps in forming the thin film lining the passage. The conductive contacts and the thin resistive film may therefore be formed more or less concurrently.

Contacts and conductive pathways from the heater element to the power source can be made of platinum, gold, copper, silver, aluminum or any suitable material. For example, vias extending to the heater can be formed in layer 110 and/or layer 120 and the vias can be filled with conductive material to form the contacts. Alternatively as shown in FIG. 5, the vias 193 may not be in direct communication with the fluid passage 130, rather vias 193 can connect external contacts 191 to conductive circuit elements 197 printed on an interior surface of a first layer 110 and/or a second layer 120 which provide electrical connections to the fluid passage 130 and the resistive heater to be formed therein. Techniques applicable for forming contacts in low temperature cofired ceramic (LTCC) applications, can be used for forming the vias, contacts and other electrical connections. Contacts may be positioned before assembly of the passage, and/or before formation of the thin resistive film heater. Alternatively, inlet and/or outlet fittings of conductive material, or coated with a conductive material, may be bonded to the interior surface of the fluid passage. However, mechanical connections can be used to provide the electrical connections provided such connections can withstand heating and cooling cycles of the heater. Techniques such as diffusive bonding are preferred in order to achieve secure attachment of the contacts of the substrate. Conductive leads attached to exposed conductive surfaces of fittings can be used to supply power to the interior heater.

A single fluid passage with a single heater or multiple heater configurations may be used. Multiple heater configurations may be constructed for example by connecting, in series, individually formed fluid passages, each including a separate heater. The use of multiple heaters may enable fluid in serially connected fluid passages 130 to be maintained at different temperatures. Such differing temperature zones in connected fluid passages may be useful in fluid temperature control devices, as discussed in U.S. application Ser. No. 09/742,322, filed Dec. 22, 2000, the entire contents of which document are incorporated by reference herein.

The device 100 may generate a vaporized fluid on an intermittent or continuous basis. For intermittent generation of an aerosol, for example, the fluid supply 150 provides the fluid in liquid phase to the fluid passage 130 each time the generation of an aerosol is desired. The valve 160 and/or the pump 162 may be used to actuate the flow of fluid from the liquid supply 150 to the fluid passage 130. As the fluid is vaporized, the remaining fluid in liquid phase between the liquid supply 150 and the fluid passage 130 can be prevented from traveling back into the liquid supply 150 by any suitable device such as the valve 160 and/or the pump 162.

For generating an intermittent aerosol in inhalation applications, the aerosol generator 100 is preferably provided with a puff-actuated sensor 144, which is preferably arranged inside a mouthpiece 142 disposed proximate to the outlet 140 as seen in FIGS. 3-4. The puff-actuated sensor 144 can be used to actuate the valve 160 and/or the pump 162 and the heater 180 so that the fluid supply 150 provides the fluid in liquid phase to the fluid passage 130, and the fluid is volatilized by the heater 180. The puff-actuated sensor 144 is preferably sensitive to pressure drops occurring in the mouthpiece 142 when a user draws on the mouthpiece 142. The aerosol generator 100 is preferably provided with circuitry such that, when a user draws on the mouthpiece 142, the valve 160 and/or pump 162 supply fluid in liquid phase to the fluid passage 130 and the heater 180 is heated by the power supply 190.

A puff-actuated sensor 144 suitable for use in the aerosol generator 100 includes, for example, Model 163PC01D35 silicon sensor, manufactured by the MicroSwitch division of Honeywell, Inc., located in Freeport, Ill., or SLP004D 0-4″ H₂O Basic Sensor Element, manufactured by SenSym, Inc., located in Milpitas, Calif. Other known flow-sensing devices, such as those using hot-wire anemometry principles, may also be suitable for use with the aerosol generator 100.

As shown in FIG. 6, a fluid passage incorporating a heater according to the invention is not limited to an assembly of layers of ceramic substrate. For example, in an alternative embodiment, the fluid passage 230 is formed in a tubular arrangement 210 which can comprise for example a glass, ceramic, or polymer tubing, or a material such as a stainless steel tube coated on the interior with a non-conductive material. A heater according to the invention may be deposited within the passage 230 by any of the techniques described above. Inlet and outlet fittings

may be constructed of a conductive material or plated so as to conduct electricity to a thin resistive film heater deposited within the passage 230. Alternatively, the fittings 280, 290 are optional and instead vias may be formed in the wall of tube 210 and filled with electrically conductive material as discussed above or printed circuits may conduct electrical power from contact points on the surface of the passage assembly 210 to the film heater lining the fluid passage 230. An aerosol generator 300 incorporating a heater formed inside a fluid passage 230 may further comprise a fluid reservoir 150, valve 160, pump 162, control arrangement 170, and power supply 190 as described above.

While the invention has been described in detail with reference to preferred embodiments thereof, it will be apparent to one skilled in the art that various changes can be made, and equivalents employed, without departing from the scope of the invention.

What is claimed is:

1. A fluid vaporizing device, comprising:
a fluid source;
a fluid passage through which fluid from the fluid source is vaporized; and
a tubular heater comprising a thin electrically resistive film lining the interior surface of all or part of the length of the fluid passage.

2. The fluid vaporizing device of claim 1, wherein the heater comprises one or more of platinum, gold, nickel, silver, or tin in the form of a pure metal, alloy, mixture, or plural layers and/or the fluid passage is of capillary size.

3. The fluid vaporizing device of claim 1, wherein the fluid passage is located in a monolithic or multilayer body of an electrically insulating material and/or the fluid passage has a uniform cross section along the length thereof, and a maximum width of the fluid passage is 0.01 to 10 mm.

4. The fluid vaporizing device of claim 1, wherein the heater comprises a deposited layer of platinum.

5. The fluid vaporizing device of claim 1, wherein the heater is a bio-compatible material, and the heater is arranged to be in direct contact with fluid in the passage.

6. The fluid vaporizing device of claim 1, wherein the fluid passage is at least partially defined by first and second layers of material enclosing a channel therebetween, or wherein the fluid passage is defined by a stack of first, second and third layers of material with the third layer comprising a void enclosed between the first and second layers; and, wherein the layers are assembled to form the fluid passage prior to the formation of the heater.

7. The fluid vaporizing device of claim 1, wherein the thin electrically resistive film is deposited by a process selected from the group consisting of thermally decomposing a metal salt deposited in the passage in solution, heating a metal powder deposited in the passage in suspension or emulsion in a carrier, reducing a metal oxide deposited within the passage in a suspension or emulsion in a carrier, coating the passage with resistive ink, electroless deposition of one or more layers of metal, and vapor deposition of a metal by electrically heating a wire threaded through the passage.

8. The fluid vaporizing device of claim 1, wherein the thin electrically resistive film heater has been formed within the fluid passage by steps of coating the interior of the passage with a metal powder, metal oxide powder, or metal salt in solution, suspension, or dispersion, and heating the passage to a temperature sufficient to reduce the deposited material to a thin metal film.

9. The fluid vaporizing device of claim 1, wherein the thin electrically resistive film heater has been formed within the fluid passage by steps of:
(a) coating the interior of the passage with a metal salt solution; and,
(b) heating the passage to a temperature sufficient to reduce the deposited material to a thin metal film.

10. The fluid vaporizing device of claim 1, wherein the fluid passage is located in an aerosol generator of an inhaler having a mouthpiece, the outlet of the passage directing volatilized fluid into the mouthpiece of the inhaler wherein an aerosol can be formed in the mouthpiece.

11. The fluid vaporizing device of claim 1, further comprising a power supply arranged to supply electrical current to the heater sufficient to resistively heat the heater and volatilize the fluid in the passage.

12. The fluid vaporizing device of claim 3, further comprising at least two vias filled with an electrically conductive material connecting electrical contacts on an exterior surface of the body to the heater within the interior of the fluid passage or to conductive elements in communication with the heater within the interior of the fluid passage.

13. The fluid vaporizing device of claim 1, wherein the heater is arranged to directly contact the fluid in the fluid passage or wherein the heater is coated with a material comprising glass, polymer, and/or resin.

14. The fluid vaporizing device of claim 11, further comprising a controller operably connected to the power source to allow intermittent activation of the heater.

15. A method for generating a vaporized fluid, comprising the steps of:
(a) supplying fluid to a fluid passage, wherein a heater is arranged to volatilize the fluid in the fluid passage such that the volatilized fluid is directed to an outlet from which the volatilized fluid is ejected out of the fluid passage;
(b) heating the heater so as to volatilize the fluid in the fluid passage; and
(c) directing the volatilized fluid out of the fluid passage via the outlet;
wherein the heater comprises a tubular thin resistive film lining the fluid passage.

16. The method of claim 15, wherein the fluid passage comprises a passage in a body of an inhaler and the volatilized fluid is ejected through an opening in a surface of the body so as to form an aerosol to be inhaled.

17. The method of claim 15, wherein the fluid passage has a maximum cross sectional dimension of 0.01 to 10 mm and the fluid comprises a solution containing a medicated fluid.

18. A method of manufacturing a fluid vaporizing device comprising the steps of:
(a) providing a fluid passage in a body, the fluid passage having an inlet opening and an outlet opening; and,
(b) forming a tubular heater by depositing a thin resistive film inside said fluid passage such that the film lines all or part of the length of the passage;
the heater being operable to volatilize fluid in the passage by passing an electrical current through the film.

19. The method of claim 18, wherein the depositing step comprises introducing a metal in solution, suspension, or dispersion in the flow passage and depositing metal on the interior of the passage.

20. The method of claim 18, wherein the depositing step comprises introducing a solution containing a platinum salt into the fluid passage, depositing platinum and heating the deposited platinum.

21. The method of claim 18, further comprising the step of forming conductive contacts electrically connecting the exterior of the body to the interior of the passage; the contacts being operable to supply an electrical current to the heater and wherein the contacts may be formed before, after, or concurrently with the formation of the heater.

22. The method of claim 18, wherein the depositing step comprises a process chosen from the group consisting of:
thermally decomposing a metal salt, deposited in the passage in solution, to a thin resistive metal film;
heating a metal powder deposited in the passage in suspension or emulsion;
reduction of a metal oxide deposited within the passage in a suspension or emulsion,
coating the passage with resistive ink;
electrolessly depositing of one or more layers of metal; and,
vapor depositing a metal by electrically heating a wire threaded through the passage.

23. The method of claim 18, wherein the depositing step comprises:
(a) coating the interior of the passage with a layer of metal powder, salt, or oxide in solution, suspension, or dispersion; and,
(b) heating the layer to a temperature sufficient to convert the layer to a thin metal film.

24. The method of claim 18, wherein the depositing step comprises:
(a) coating the interior of the passage with a metal salt solution; and,
(b) heating the passage to a temperature sufficient to reduce the deposited material to a thin metal film.

25. A fluid vaporizing device made by the method of claim 18.
